(19) United States
(12) Patent Application Publication
(10) Pub. No.: US 2024/0277891 A1
(43) Pub. Date: Aug. 22, 2024

(54) MEDICAL TREATMENT MATERIAL AND METHOD FOR PRODUCING SAME

(71) Applicants: Yoshiyuki Koyama, Tokyo (JP); TOAGOSEI CO., LTD., Tokyo (JP)

(72) Inventors: Yoshiyuki Koyama, Tokyo (JP); Tomoko Ito, Chiba (JP); Kenichi Nakamura, Aichi (JP); Ayaka Ouchi, Aichi (JP)

(73) Assignees: Yoshiyuki Koyama, Tokyo (JP); TOAGOSEI CO., LTD., Tokyo (JP)

(21) Appl. No.: 18/571,487

(22) PCT Filed: Jun. 27, 2022
(86) PCT No.: PCT/JP2022/025565

§ 371 Date: Dec. 18, 2023

(30) Foreign Application Priority Data
Jun. 29, 2021 (JP) 2021-108066

(51) Int. Cl. A61L 26/00; C08J 5/18

(52) U.S. Cl. CPC A61L 26/008; A61L 26/0052; C08J 5/18; C08J 2333/02; C08J 2333/10; C08J 2433/02; C08J 2439/06

(57) ABSTRACT

A medical treatment material that forms a hydrogel upon contact with water contains a polymer (A) including a structural unit derived from an ethylenic unsaturated monomer (ma) which has a carboxyl group and a molecular weight of 115 or less, and a polymer (B) including a structural unit derived from an ethylenic unsaturated monomer (mb) which has a functional group E that is capable of forming a hydrogen bond with a carboxyl group, the polymer (B) not being the polymer (A). At least one of the polymer (A) and the polymer (B) includes a structural unit derived from an ethylenic unsaturated monomer (mc) differing from the ethylenic unsaturated monomers (ma) and (mb), and the monomer (mc) is not an ethylenic unsaturated monomer having a cross-linkable functional group.

### CROSS-REFERENCE TO RELATED APPLICATIONS

The present application claims the benefit of Japanese Patent Application No. 2021-108066 filed on Jun. 29, 2021, the disclosure of which is incorporated herein by reference.

The present disclosure relates to a medical treatment material and to a method for producing the material (hereinafter may also be referred to as a “medical treatment material production method”). More particularly, the disclosure relates to a medical treatment material that forms a hydrogel upon contact with water, and to a method for producing the material.

### BACKGROUND ART

A hydrogel that can adhere to a biotissue is applicable to an anti-adhesive material (for preventing synechia), a hemostatic material, a wound dressing material, or the like. Hitherto, various studies have been conducted on such a hydrogel (see, for example, Patent Document 1). In Patent Document 1, a hydrogel-forming material is proposed. The material is a medical treatment material which is capable of forming a hydrogel via a hydrogen bond between poly(acrylic acid) and polyvinylpyrrolidone. According to the technique disclosed in Patent Document 1, an aqueous solution of any one of poly(acrylic acid) and polyvinylpyrrolidone is dried to form a film, and another aqueous solution of the counter component is brought into contact with the film, followed by drying, to thereby yield a hydrogel-forming material. The material is in the form of dry film or sponge which is capable of forming a hydrogel upon absorption of water. The thus-obtained film and sponge can rapidly absorb a water content of blood, tissue fluid, etc. present on a wet biotissue (e.g., a wound or a hemostatic part), to thereby swell. As a result, the material can adhere to a biotissue.

### PRIOR ART DOCUMENTS

### Patent Documents

Patent Document 1: Japanese Patent Application Laid-Open (kokai) No. 2014-100462

### SUMMARY OF THE INVENTION

### Problems to be Solved by the Invention

In one previous investigation conducted by the present inventors, when a hydrogel-forming material disclosed in Patent Document 1 swelled through contact with water, a sufficient adhesion property to a biotissue was not completely achieved. Thus, in the case where the hydrogel-forming material is attached to a wet biotissue, the formed hydrogel may possibly detach itself from the biotissue.

The present disclosure has been made under the above circumstances. Thus, an object of the disclosure is to provide a medical treatment material which provides an excellent adhesion property to a biotissue.

### Means for Solving the Problems

The present inventors have carried out extensive studies in order to solve the aforementioned problem, and have found that a medical treatment material which contains a specific polymer having a carboxyl group and another polymer which is capable of forming a hydrogen bond with the specific polymer exhibits high adhesion performance with respect to a biotissue. Accordingly, the present disclosure provides the following specific means.
[1] A medical treatment material that forms a hydrogel upon contact with water, which contains a polymer (A) including a structural unit derived from an ethylenic unsaturated monomer (ma) which has a carboxyl group and a molecular weight of 115 or less, and a polymer (B) including a structural unit derived from an ethylenic unsaturated monomer (mb) which has a functional group E that is capable of forming a hydrogen bond with a carboxyl group, the polymer (B) not being the polymer (A), wherein at least one of the polymer (A) and the polymer (B) includes a structural unit derived from an ethylenic unsaturated monomer (mc) differing from the ethylenic unsaturated monomers (ma) and (mb), the monomer (mc) is not an ethylenic unsaturated monomer having a cross-linkable functional group.
[2] The medical treatment material of [1] above, wherein the ethylenic unsaturated monomer (mc) includes an ethylenic unsaturated monomer (mc1) which has a carboxyl group and a molecular weight of more than 115.
[3] The medical treatment material of [2] above, wherein the ethylenic unsaturated monomer (mc1) is at least one species selected from the group consisting of an acrylic acid dimer, an acrylic acid trimer, an acrylic acid tetramer, and ω-carboxy-caprolactone mono(meth)acrylate.
[4] The medical treatment material of [1] to [3] above, wherein the ethylenic unsaturated monomer (mc) includes an ethylenic unsaturated monomer (mc2) which has no carboxyl group and no functional group E.
[5] The medical treatment material of [1] to [4] above, wherein the polymer (A) includes a structural unit derived from the ethylenic unsaturated monomer (mc), and has a ratio by mass ((ma)/(mc)) of the ethylenic unsaturated monomer (ma) to the ethylenic unsaturated monomer (mc), each forming the polymer (A), of 99.7/0.3 to 50/50.
[6] The medical treatment material of [1] to [5] above, wherein the polymer (A) is a cross-linked polymer.
[7] The medical treatment material of [1] to [6] above, wherein the polymer (B) has an amide group.
[8] The medical treatment material of [1] to [7] above, wherein the polymer (B) is at least one species selected from the group consisting of polyvinylpyrrolidone, polyacrylamide, and polymethacrylamide.
[9] The medical treatment material of [1] to [8] above, which is in a state where a film-shape solid containing one of the polymer (A) and the polymer (B) has been brought into contact with a solution containing the other of them and the contact product is dried; which forms a hydrogel upon absorption of water, and which exhibits an adhesion property to biotissue.
[10] A method for producing a medical treatment material for forming a hydrogel upon contact with water, the production method comprising a step of preparing a film-shape solid containing one of a polymer (A) and a polymer (B), and a step of bringing a solution containing the other of the polymer (A) and polymer (B) into contact with the film-shape solid, followed by drying, wherein the polymer (A) is a polymer including a structural unit derived from an ethylenic unsaturated monomer (ma) which has a carboxyl group and a molecular weight of 115 or less; the polymer (B) is a polymer including a structural unit derived from an ethylenic unsaturated monomer (mb) which has a functional group E that is capable of forming a hydrogen bond with a carboxyl group, the polymer (B) not being the polymer (A); and at least one of the polymer (A) and the polymer (B) includes a structural unit derived from an ethylenic unsaturated monomer (mc) differing from the ethylenic unsaturated monomers (ma) and (mb), the monomer (mc) is not an ethylenic unsaturated monomer having a cross-linkable functional group.

### Advantageous Effects of the Invention

According to the present disclosure, polymer components of a medical treatment material that is capable of forming a hydrogel are provided in the following manner. Specifically, a polymer (A) including a structural unit derived from an ethylenic unsaturated monomer (ma) which has a carboxyl group and a molecular weight of 115 or less, and a polymer (B) including a structural unit derived from an ethylenic unsaturated monomer (mb) which has a functional group E that is capable of forming a hydrogen bond with a carboxyl group are combined, and at least one of the polymer (A) and the polymer (B) is provided as a polymer including a structural unit derived from an ethylenic unsaturated monomer (mc). As a result, a medical treatment material exhibiting an excellent adhesion property to a biotissue can be obtained.

### MODES FOR CARRYING OUT THE INVENTION

The present disclosure will next be described in detail. Notably, in the present specification, the expression “(meth)acrylic” refers to “acrylic and/or methacrylic.” The expression “(meth)acrylate” refers to “acrylate and/or methacrylate.”

### <<<Medical Treatment Material>>

The medical treatment material of the present disclosure is a medical treatment material that is capable of forming a hydrogel upon contact with water. The medical treatment material is a product for forming a hydrogel applicable to an anti-adhesive material, a hemostatic material, a wound dressing material, or the like. Specific examples thereof include hydrogel-forming materials in the form of film, sponge, sheet, or powder. The medical treatment material of the present disclosure contains the following polymers (A) and (B):
Polymer (A): polymer (A) including a structural unit derived from an ethylenic unsaturated monomer (ma) which has a carboxyl group and a molecular weight of 115 or less, and
Polymer (B): polymer (B) including a structural unit derived from an ethylenic unsaturated monomer (mb) which has a functional group E that is capable of forming a hydrogen bond with a carboxyl group (the polymer (B) not being the polymer (A)).

Also, among the polymer (A) and the polymer (B) contained in the medical treatment material of the present disclosure, at least one of the polymer (A) and the polymer (B) includes a structural unit derived from an ethylenic unsaturated monomer (mc) differing from the ethylenic unsaturated monomers (ma) and (mb). The monomer (mc) is not an ethylenic unsaturated monomer having a cross-linkable functional group. Hereinafter, the components contained in the medical treatment material of the present disclosure will be described.

### <Polymer (A)>

(Ethylenic Unsaturated Monomer (ma))

Examples of the ethylenic unsaturated monomer (ma) serving as a component of the polymer (A) include (meth)acrylic acid, crotonic acid, and 2-ethylpropenic acid. Among ethylenic unsaturated monomers (ma), (meth)acrylic acid is preferred. Acrylic acid is more preferred, since it can form, upon contact with water, a hydrogel exhibiting a higher adhesion property to a biotissue.

In the polymer (A), the relative amount of the structural unit derived from the ethylenic unsaturated monomer (ma) with respect to all the structural units forming the polymer (A) is preferably 40 mass % or more, more preferably 50 mass % or more, still more preferably 60 mass % or more, yet more preferably 70 mass % or more, particularly preferably 80 mass % or more. Also, the relative amount of the structural unit derived from the ethylenic unsaturated monomer (ma) with respect to all the structural units forming the polymer (A) is preferably 99.9 mass % or less, more preferably 99.7 mass % or less, still more preferably 99.5 mass % or less. When the amount of the structural unit derived from the ethylenic unsaturated monomer (ma) in the polymer (A) satisfies the above conditions, a hydrogel exhibiting a higher adhesion property to a biotissue can be formed, which is preferred. Notably, the ethylenic unsaturated monomer (ma) forming the polymer (A) may be used singly or in combination of two or more species.

### (Ethylenic Unsaturated Monomer (Mc))

In addition to the ethylenic unsaturated monomer (ma), the polymer (A) preferably includes a structural unit derived from the ethylenic unsaturated monomer (mc). By incorporating a structural unit derived from the ethylenic unsaturated monomer (mc) into the polymer (A), the adhesion property to a biotissue can be enhanced.

No particular limitation is imposed on the ethylenic unsaturated monomer (mc), so long as the monomer has only one ethylenic unsaturated group involved in polymerization and can be co-polymerized with the ethylenic unsaturated monomer (ma). Examples of the ethylenic unsaturated monomer (mc) include an ethylenic unsaturated monomer which has a carboxyl group and a molecular weight of more than 115 (hereinafter may also be referred to as “unsaturated monomer (mc1)”), and an ethylenic unsaturated monomer which has no carboxyl group and no functional group E (hereinafter may also be referred to as “unsaturated monomer (mc2)”).

Unsaturated Monomer (mc1)

Examples of the unsaturated monomer (mc1) include maleic acid, fumaric acid, itaconic acid, citraconic acid, cinnamic acid, a (meth)acrylic acid dimer, a (meth)acrylic acid trimer, a (meth)acrylic acid tetramer, mono(2-(meth)acryloyloxyethyl) succinate, monohydroxyethyl (meth)acrylate phthalate, ω-carboxy-caprolactone mono(meth)acrylate, and 4-carboxystyrene. From the viewpoint of a high effect of improvement in adhesion to a biotissue, among unsaturated monomers (mc1), at least one species selected from the group consisting of an acrylic acid dimer, an acrylic acid trimer, an acrylic acid tetramer, and ω-carboxy-caprolactone mono(meth)acrylate is preferably used.

Unsaturated Monomer (mc2)

Examples of the unsaturated monomer (mc2) include alkyl (meth)acrylate ester, alicyclic (meth)acrylate ester, aromatic (meth)acrylate ester, alkoxyalkyl (meth)acrylate ester, hydroxyalkyl (meth)acrylate ester, and polyalkylene glycol mono(meth)acrylate.

From the viewpoint of securing water-solubility of the polymer (A), the alkyl (meth)acrylate ester is preferably a compound in which the alkyl group (R) of the alkyl ester moiety (—COOR) has 1 to 12 carbon atoms. Specific examples include methyl (meth)acrylate, ethyl (meth)acrylate, isopropyl (meth)acrylate, n-propyl (meth)acrylate, n-butyl (meth)acrylate, isobutyl (meth)acrylate, tert-butyl (meth)acrylate, hexyl (meth)acrylate, 2-ethylhexyl (meth)acrylate, n-octyl (meth)acrylate, isooctyl (meth)acrylate, n-nonyl (meth)acrylate, isononyl (meth)acrylate, decyl (meth)acrylate, and dodecyl (meth) acrylate.

Specific examples of the alicyclic (meth)acrylate ester include cyclohexyl (meth)acrylate, methylcyclohexyl (meth)acrylate, tert-butylcyclohexyl (meth)acrylate, cyclododecyl (meth)acrylate, isobornyl (meth)acrylate, adamantyl (meth)acrylate, dicyclopentenyl (meth)acrylate, and dicyclopentanyl (meth)acrylate. Specific examples of the aromatic (meth)acrylate ester include phenyl (meth)acrylate, benzyl (meth)acrylate, phenoxymethyl (meth)acrylate, 2-pnenoxyethyl (meth)acrylate, and 3-phenoxypropyl (meth) acrylate.

Specific examples of the alkoxyalkyl (meth)acrylate ester include methoxyethyl (meth)acrylate, ethoxyethyl (meth)acrylate, n-propoxyethyl (meth)acrylate, n-butoxyethyl (meth)acrylate, methoxypropyl (meth)acrylate, ethoxypropyl (meth)acrylate, n-propoxypropyl (meth)acrylate, n-butoxypropyl (meth)acrylate, methoxybutyl (meth)acrylate, ethoxybutyl (meth)acrylate, n-propoxybutyl (meth)acrylate, and n-butoxybutyl (meth)acrylate.

Specific examples of the hydroxyalkyl (meth)acrylate ester include 2-hydroxyethyl (meth)acrylate, 2-hydroxypropyl (meth)acrylate, 3-hydroxypropyl (meth)acrylate, 2-hydroxybutyl (meth)acrylate, 3-hydroxybutyl (meth)acrylate, and 4-hydroxybutyl (meth)acrylate. Examples of the polyalkylene glycol mono(meth)acrylate include polyethylene glycol mono(meth)acrylate, polypropylene glycol mono(meth)acrylate, and polyethylene glycol-polypropylene glycol mono(meth)acrylate.

Among the above specific monomers, the unsaturated monomer (mc2) is preferably at least one species selected from the group consisting of alkyl (meth)acrylate esters, alicyclic (meth)acrylate esters, aromatic (meth)acrylate ester, and alkoxyalkyl (meth)acrylate esters. Among them, the unsaturated monomer (mc2) is preferably an alkyl (meth)acrylate ester, from the viewpoint of a high effect of improvement in adhesion to a biotissue. Particularly, the alkyl (meth)acrylate ester forming the polymer (A) is preferably a compound in which the alkyl group (R) has 1 to 8 carbon atoms, more preferably a compound in which the alkyl group (R) has 1 to 4 carbon atoms. Notably, the alkyl (meth)acrylate ester forming the polymer (A) may be used singly or in combination of two or more species.

When the polymer (A) includes a structural unit derived from the ethylenic unsaturated monomer (mc), the ratio by mass ((ma)/(mc)) of the ethylenic unsaturated monomer (ma) to the ethylenic unsaturated monomer (mc), each forming the polymer (A), is preferably 99.7/0.3 to 50/50. A ratio by mass ((ma)/(mc)) of the ethylenic unsaturated monomer (ma) to the ethylenic unsaturated monomer (mc) falling within the above range is preferred, since the formed hydrogel exhibits a high effect of improvement in adhesion to a biotissue. From this viewpoint, the ratio by mass ((ma)/(mc)) of the ethylenic unsaturated monomer (ma) to the ethylenic unsaturated monomer (mc), each forming the polymer (A), is more preferably 99.7/0.3 to 60/40, still more preferably 99.5/0.5 to 70/30, yet more preferably 99.5/0.5 to 80/20. Notably, the ethylenic unsaturated monomer (mc) forming the polymer (A) may be used singly or in combination of two or more species.

Particularly when the unsaturated monomer (mc1) is used as the ethylenic unsaturated monomer (mc), the effect of improvement in adhesion of a hydrogel to a biotissue can be fully attained by a smaller amount of the ethylenic unsaturated monomer (mc) incorporated into the polymer (A) (i.e., by a larger amount of the ethylenic unsaturated monomer (ma) incorporated thereinto). Therefore, such a mode is preferred.

The polymer (A) may be a cross-linked polymer or a polymer having a weight average molecular weight of 100,000 or more (hereinafter may also be referred to as a “high-molecular weight polymer (AH)”). Among cross-linked polymers and high-molecular weight polymers (AH), a cross-linked polymer is preferably used as the polymer (A), since cross-linked polymers are more excellent in swellability upon contact with water and adhesion to a biotissue. A cross-linked polymer serving as the polymer (A) is preferably used, when the polymer (B) includes a structural unit derived from the ethylenic unsaturated monomer (mc).

When the polymer (A) is a cross-linked polymer, the polymer (A) preferably includes a structural unit derived from an ethylenic unsaturated monomer having a cross-linkable functional group (hereinafter may also be referred to as an “unsaturated monomer (md)”). The cross-linkable functional group present in the unsaturated monomer (md) is preferably a polymerizable unsaturated group or a self-cross-linkable functional group. Specific examples of the unsaturated monomer (md) include a multi-functional polymerizable monomer having two or more ethylenic unsaturated groups, and a self-cross-linkable monomer having a self-cross-linkable functional group (e.g., a hydrolyzable silyl group). Specific examples of the multi-functional polymerizable monomer include a multi-functional (meth)acrylate compound, a multi-functional alkenyl compound, and a compound having both a (meth)acryloyl group and an alkenyl group. Amon them, the ethylenic unsaturated monomer having a cross-linkable functional group is preferably a multi-functional alkenyl compound, from the viewpoint of easily forming a uniform cross-link structure.

Specific examples of the multi-functional alkenyl compound include multi-functional allyl ether compounds such as trimethylolpropane diallyl ether, trimethylolpropane triallyl ether, pentaerythritol diallyl ether, pentaerythritol triallyl ether, tetraallyoxyethane, and polyallysaccharose; multi-functional allyl compounds such as diallyl phthalate; multi-functional vinyl compounds such as divinylbenzene; and alkenyl group-containing (meth)acrylic acid compounds such as allyl (meth)acrylate, isopropenyl (meth)acrylate, butenyl (meth)acrylate, pentenyl (meth)acrylate, and 2-(2-vinyloxyethoxy)ethyl (meth)acrylate. Of these, a multi-functional allyl ether compound having a plurality of allyl ether groups in the molecule thereof is particularly preferred as the multi-functional alkenyl compound.

Specific examples of the self-cross-linkable monomer include a vinyl monomer having a hydrolyzable silyl group. Examples of the vinyl monomer having a hydrolyzable silyl group include vinyl silanes such as vinyltrimethoxysilane, vinyltriethoxysilane, vinylmethyldimethoxysilane, and vinyldimethylmethoxysilane; silyl group-containing (meth)acrylate esters such as trimethoxysilylpropyl (meth)acrylate, triethoxysilylpropyl (meth)acrylate, and methyldimethoxysilylpropyl (meth)acrylate; trimethoxysilylpropyl vinyl ether; and vinyl trimethoxysilylundecanate.

When the polymer (A) includes a structural unit derived from the unsaturated monomer (md), the relative amount of the structural unit derived from unsaturated monomer (md) included in the polymer (A) with respect to all the structural units forming the polymer (A) is preferably 0.01 mass % or more, more preferably 0.1 mass % or more. Also, the relative amount of the structural unit with respect to all the structural units forming the polymer (A) is preferably 5 mass % or less, more preferably 2 mass % or less, still more preferably 1 mass % or less. Notably, the unsaturated monomer (md) forming the polymer (A) may be used singly or in combination of two or more species.

When a cross-linked polymer is used as the polymer (A), a commercial product thereof may be used. Examples of the commercial product include products (as tradenames) such as JUNLON (registered trademark) PW-120, JUNLON PW-121, and JUNLON PW-312S (products of TOAGOSEI Co., Ltd.); and Carbopol (registered trademark) 934P NF, Carbopol 981, Carbopol Ultrez10, and Carbopol Ultrez30 (products of Lubrizol).

When a high-molecular weight polymer (AH) is used as the polymer (A), the high-molecular weight polymer (AH) is preferably a polymer having no structural unit (mc1). No particular limitation is imposed on the weight average molecular weight (Mw) of the high-molecular weight polymer (AH). From the viewpoint of securing dynamic strength and thickening effect, the molecular weight is preferably 5×10⁵ or more, more preferably 1×10⁶ or more, still more preferably 1.8×10⁶ or more. From the viewpoint of ease of handling, the molecular weight of high-molecular weight polymer (AH) is preferably 1×10⁷ or less, more preferably 8×10⁶ or less, still more preferably 5×10⁶ or less. Meanwhile, the molecular weight of the high-molecular weight polymer (AH) is a value obtained by methylating a carboxyl group with trimethylsilyldiazomethane, measuring through gel permeation chromatography (GPC) with tetrahydrofuran as an eluent, and reducing the measurement to polystyrene.

### <Polymer (B)>

No particular limitation is imposed on the polymer (B), so long as the polymer (B) has a functional group E that is capable of forming a hydrogen bond with a carboxyl group included in the polymer (A), and is a polymer different from the polymer (A). Examples of the functional group E include an amide group, a cyano group, a carbonyl group, an amino group, and a hydroxy group. The functional group E in the polymer (B) may be a single species or a multi-species of two or more species.

By forming a hydrogen bond between a carboxyl group of the polymer (A) and the functional group E, a hydrogel-forming material having excellent water-swelling property can be produced. In this viewpoint, the functional group E is, among others, preferably an amide group and/or a hydroxy group, with an amide group being particularly preferred.

Preferably, the polymer (B) having an amide group is produced through polymerization using an ethylenic unsaturated monomer having an amide group as the ethylenic unsaturated monomer (mb). Examples of the ethylenic unsaturated monomer having an amide group include (meth)acrylamide, N,N-dimethyl(meth)acrylamide, N,N-dimethylaminopropyl(meth)acrylamide, N-methyl(meth)acrylamide, N-vinyl-2-pyrrolidone, and 1-vinyl-4-methyl-2-pyrrolidone.

Examples of the polymer (B) having a hydroxy group include polyethylene glycol (e.g., Macrogol 4000, Macrogol 6000, and Macrogol 20000, commercial products of NOF Corporation); polyoxyethylene-hardened castor oil (e.g., Cremophor RH40, commercial product of BASF, and HCO-40 and HCO-60, commercial products of Nikko Chemicals, Co., Ltd.); polyoxyethylene polyoxypropylene glycol (e.g., Pluronic (registered trademark) F68, commercial product of ADEKA); and poly(vinyl alcohol). Of these, the polymer (B) having a hydroxy group is preferably polyethylene glycol.

In the polymer (B), the relative amount of the structural unit derived from the ethylenic unsaturated monomer having a functional group E with respect to all the structural units forming the polymer (B) is preferably 70 mass % or more, more preferably 80 mass % or more, still more preferably 90 mass % or more, yet more preferably 97 mass % or more.

As the polymer (B), a cross-linked polymer or a polymer having a weight average molecular weight of 10,000 or more (hereinafter may also be referred to as a “high-molecular weight polymer (BH)” is preferably used. More preferably, a high-molecular weight polymer (BH) is used.

From the viewpoint of producing a medical treatment material that can form a hydrogel exhibiting high water-swellability upon contact with water, the polymer (B) is preferably at least one species selected from the group consisting of polyvinylpyrrolidone, polyacrylamide, and polymethacrylamide. From the viewpoint of excellent polymerization performance of component monomers and ease of production of the polymer (B), the polymer (B) is more preferably at least one species selected from the group consisting of polyvinylpyrrolidone and polyacrylamide.

In addition to the ethylenic unsaturated monomer (mb), the polymer (B) may include a structural unit derived from the ethylenic unsaturated monomer (mc). When the polymer (B) further includes a structural unit derived from the ethylenic unsaturated monomer (mc), adhesion property to a biotissue can be enhanced, which is preferred. Specific examples of the ethylenic unsaturated monomer (mc) includes the same monomers as exemplified in relation to the polymer (A).

When the polymer (B) includes the ethylenic unsaturated monomer (mc), the ratio by mass ((mb)/(mc)) of the ethylenic unsaturated monomer (mb) to the ethylenic unsaturated monomer (mc), each forming the polymer (B), is preferably 99.7/0.3 to 50/50. A ratio by mass ((mb)/(mc)) of the ethylenic unsaturated monomer (mb) to the ethylenic unsaturated monomer (mc) falling within the above range is preferred, since the formed hydrogel exhibits a high effect of improvement in adhesion to a biotissue. The ratio by mass ((mb)/(mc)) of the ethylenic unsaturated monomer (mb) to the ethylenic unsaturated monomer (mc), each forming the polymer (B), is more preferably 99.7/0.3 to 60/40, still more preferably 99.5/0.5 to 70/30, yet more preferably 99.5/0.5 to 80/20. Notably, the ethylenic unsaturated monomer (mc) forming the polymer (B) may be used singly or in combination of two or more species. In consideration of low co-polymerizability between the ethylenic unsaturated monomer (mb) and the ethylenic unsaturated monomer (mc), the polymer (A) preferably includes a structural unit derived from the ethylenic unsaturated monomer (mc).

Polyvinylpyrrolidone serving as the polymer (B) is typically a polymer of N-vinyl-2-pyrrolidone and may further include a structural unit derived from the ethylenic unsaturated monomer (mc). So long as the effects of the present disclosure are not impaired, polyvinylpyrrolidone serving as the polymer (B) may further include a structural unit derived from a monomer differing from N-vinyl-2-pyrrolidone or the ethylenic unsaturated monomer (mc) (e.g., an unsaturated monomer (md)). In polyvinylpyrrolidone, the relative amount of the structural unit derived from a monomer differing from N-vinyl-2-pyrrolidone or the ethylenic unsaturated monomer (mc), with respect to all the structural units forming polyvinylpyrrolidone, is preferably 3 mass % or less, more preferably 1 mass % or less.

Similarly, polyacrylamide serving as the polymer (B) is typically a polymer of acrylamide and may further include a structural unit derived from the ethylenic unsaturated monomer (mc). So long as the effects of the present disclosure are not impaired, polyacrylamide serving as the polymer (B) may further include a structural unit derived from a monomer differing from acrylamide or the ethylenic unsaturated monomer (mc) (e.g., an unsaturated monomer (md)). In polyacrylamide, the relative amount of the structural unit derived from a monomer differing from acrylamide or the ethylenic unsaturated monomer (mc), with respect to all the structural units forming polyacrylamide, is preferably 3 mass % or less, more preferably 1 mass % or less.

Polymethacrylamide serving as the polymer (B) is typically a polymer of methacrylamide and may further include a structural unit derived from the ethylenic unsaturated monomer (mc). So long as the effects of the present disclosure are not impaired, polymethacrylamide serving as the polymer (B) may further include a structural unit derived from a monomer differing from methacrylamide or the ethylenic unsaturated monomer (mc) (e.g., an unsaturated monomer (md)). In polymethacrylamide, the relative amount of the structural unit derived from a monomer differing from methacrylamide or the ethylenic unsaturated monomer (mc), with respect to all the structural units forming polymethacrylamide, is preferably 3 mass % or less, more preferably 1 mass % or less.

When a high-molecular weight polymer (BH) is used as the polymer (B), no particular limitation is imposed on the weight average molecular weight (Mw) of the high-molecular weight polymer (BH). From the viewpoint of securing dynamic strength and thickening effect, the molecular weight is preferably 1×10⁴ or more, more preferably 3×10⁴ or more, still more preferably 5×10⁴ or more. From the viewpoint of ease of handling, the molecular weight (Mw) of the high-molecular weight polymer (BH) is preferably 1×10⁸ or less, more preferably 5×10⁷ or less, still more preferably 3×10⁷ or less. Notably, the molecular weight of the polymer (B) is a molecular weight determined through gel permeation chromatography (GPC) and reduced to polystyrene.

From the viewpoints of formation of a hydrogel exhibiting an excellent adhesion property to a biotissue and provision of a medical treatment material having high dynamic strength, the total amount of the polymer (A) and the polymer (B) contained in the medical treatment material of the present disclosure with respect to the total amount of the medical treatment material is preferably 70 mass % or more, more preferably 80 mass % or more, still more preferably 90 mass % or more, yet more preferably 95 mass % or more.

In the medical treatment material of the present disclosure, the amounts of the polymer (A) and the polymer (B) content with respect to 100 parts by mass of the polymer (A) are preferably regulated such that the polymer (B) content is adjusted to 20 to 500 parts by mass. When the amounts of the polymer (A) and the polymer (B) fall within the above range, a hydrogel which contributes to a high effect of improving dynamic strength and which exhibits an excellent adhesion property to a biotissue is formed, which is preferred. From the above viewpoint, the polymer (A) content and the polymer (B) content are preferably adjusted such that the amount of the polymer (B) is more preferably 30 to 400 parts by mass, still more preferably 50 to 300 parts by mass, with respect to 100 parts by mass of the polymer (A).

No particular limitation is imposed on the polymerization method for producing the polymer (A) and the polymer (B). Actually, the polymer (A) and the polymer (B) may be produced by polymerizing monomers through, for example, a known radical polymerization technique such as solution polymerization, suspension polymerization, emulsion polymerization, or bulk polymerization. In one possible mode of the solution polymerization, an organic solvent and monomers are fed to a reactor, and a polymerization initiator (e.g., an azo compound) is added thereto. The mixture is heated at 40 to 250° C. for polymerization, to thereby yield a target polymer.

Examples of the modes of combination of the polymer (A) and the polymer (B) contained in the medical treatment material of the present disclosure are as follows:
(1) Polymer (A): a polymer including a structural unit derived from the ethylenic unsaturated monomer (ma) and a structural unit derived from the ethylenic unsaturated monomer (mc), and polymer (B): a polymer including a structural unit derived from the ethylenic unsaturated monomer (mb);
(2) Polymer (A): a polymer including a structural unit derived from the ethylenic unsaturated monomer (ma), and polymer (B): a polymer including a structural unit derived from the ethylenic unsaturated monomer (mb) and a structural unit derived from the ethylenic unsaturated monomer (mc); and
   (3) Polymer (A): a polymer including a structural unit derived from the ethylenic unsaturated monomer (ma) and a structural unit derived from the ethylenic unsaturated monomer (mc), and polymer (B): a polymer including a structural unit derived from the ethylenic unsaturated monomer (mb) and a structural unit derived from the ethylenic unsaturated monomer (mc).

Of these, the above modes (1) and (3) are preferred, from the viewpoint of yielding a hydrogel-forming material excellent in adhesion to a biotissue. From the viewpoints of co-polymerization performance between the ethylenic unsaturated monomer (ma) and the ethylenic unsaturated monomer (mb) and adhesion of the hydrogel-forming material to a biotissue, the above mode (1) is particularly preferred. In the above mode (1), a high-molecular weight polymer (BH) serving as the polymer (B) is further preferred. Notably, no precise reason has been elucidated why the adhesion property of the hydrogel-forming material to a biotissue can be enhanced by incorporating a structural unit derived from ethylenic unsaturated monomer (mc) into at least one of the polymer (A) and the polymer (B). However, one conceivable reason is that the flexibility (softness) of the hydrogel-forming material in the presence of water would be enhanced by incorporation of the structural unit derived from the ethylenic unsaturated monomer (mc).

### <Additional Components>

The medical treatment material of the present disclosure may further contain a component differing from the polymer (A) and the polymer (B) (hereinafter may also be referred to as an “additional component”) in accordance with the purpose of use and the like. Examples of the additional component include various drugs such as an antiseptic, an anti-inflammatory agent, an anti-coagulant, a local anesthetic, a vasoconstrictor, and a vasodilator; and a water-soluble polymer (C) differing from the polymer (A) and the polymer (B). The additional component may be added singly or in combination of a plurality of species. So long as the effects of the present invention are not impaired, the amount of each additional component may be appropriately tuned.

Examples of the water-soluble polymer (C) include water-soluble polymers which are generally used as a thickener. Specific examples include a polysaccharide. Examples of the polysaccharide include cellulose derivatives such as hydroxyethylcellulose, carboxymethylcellulose, and hydroxypropylmethylcellulose; mucosaccharides such as hyalurnonic acid and chondroitin sulfate; water-soluble natural high-molecule polysaccharides such as carrageenan, pectin, locust bean gum, guar gum, xanthan gum, and gellan gum; and a salt thereof (e.g., a sodium salt). Of these, the water-soluble polymer (C) is preferably hyaluronic acid or a salt thereof. The number average molecular weight of the water-soluble polymer (C) is, for example, 200,000 or more. The molecular weight of the water-soluble polymer (C) is determined through GPC and reduced to polystyrene.

When the medical treatment material of the present disclosure contains the water-soluble polymer (C), the relative amount of the water-soluble polymer (C), with respect to the total amount of the polymer (A) and the polymer (B) as 100 parts by mass, is preferably adjusted to 0.01 to 50 parts by mass. By tuning the water-soluble polymer (C) content to fall within the above range, water retentivity of the hydrogel can be improved. From this viewpoint, the water-soluble polymer (C) content with respect to the total amount of the polymer (A) and the polymer (B) as 100 parts by mass is preferably 0.1 parts by mass or more, more preferably 0.5 parts by mass or more. The upper limit of the water-soluble polymer (C) content with respect to the total amount of the polymer (A) and the polymer (B) as 100 parts by mass is more preferably 20 parts by mass or less, still more preferably 15 parts by mass or less. The water-soluble polymer (C) may be used singly or in combination of two or more species.

### <Method for Producing Medical Treatment Material>

No particular limitation is imposed on the method for producing the medical treatment material of the present disclosure, but the either of the following methods [1] and [2] is preferably employed. That is;
Method [1]: bringing a film-shape solid containing one polymer of the polymer (A) and the polymer (B) into contact with a solution containing a solution of the other polymer, followed by drying; and
Method [2]: mixing a solution containing the polymer (A) with a solution containing the polymer (B) in the presence of the water-soluble polymer (C), followed by drying.

If a solution containing the polymer (A) is simply mixed with a solution containing the polymer (B), a carboxyl group present in the polymer (A) forms a hydrogen bond with the functional group E present in the polymer (B), to thereby lead to considerably rapid formation of a hydrogel. However, the thus-formed hydrogel has an insufficient solubility and swellability in water and exhibits a poor adhesion property to a biotissue. In contrast, according to the aforementioned method [1] or [2], a medical treatment material which exhibits excellent water-solubility and water swellability can be produced.

### (Method [1])

In the method [1], firstly, a film-shape solid containing one polymer of the polymer (A) and the polymer (B) (hereinafter may also be referred to as a “first polymer”) is prepared. The film-shape solid is prepared through any of customary methods such as drying of solution and heat pressing. Of these, the solution drying method is preferred, from the viewpoints of suppression of bubble generation and formation of flat film. In one preferred mode of producing a film-shape solid through solution drying, a polymer solution is prepared by dissolving the first polymer in a solvent (hereinafter may also be referred to as a “first polymer solution”) and then the first polymer solution is applied onto a support, followed by drying. Notably, the first polymer forming the film-shape solid may be the polymer (A) or the polymer (B).

Examples of the solvent for dissolving the first polymer include water, a mixture of water and an organic solvent dissolvable in water, and an organic solvent dissolvable in water. Examples of the organic solvent dissolvable in water include methanol, ethanol, and acetone. Of these, water, ethanol, and a mixture of water and ethanol are preferred as solvents for dissolving the first polymer. No particular limitation is imposed on the polymer concentration of the first polymer solution, and it is, for example, 0.01 to 10 mass %, preferably 0.1 to 5 mass %.

No particular limitation is imposed on the method of forming the film-shape solid on a support, and any known film formation technique may be employed. In one possible procedure, the first polymer solution is applied onto a support, and the solvent is removed preferably by heating, to thereby form a film-shape solid containing the first polymer on the support. In the case where heating is conducted, the heating temperature is, for example, 50 to 120° C., and the heating time is, for example, 0.1 to 5 hours. The heating may be performed under reduced pressure or blowing. The thickness of the film-shape solid formed on a support is, for example, 1 to 5,000 m. The water content of the film-shape solid is, for example, 10 mass % or less.

Subsequently, another polymer solution (hereinafter may also be referred to as a “second polymer solution”) prepared by dissolving, in a solvent, a polymer selected from the polymer (A) and the polymer (B) which differs from the first polymer (hereinafter may also be referred to as a “second polymer”) is brought into contact with the film-shape solid formed on the support. Examples of the solvent for dissolving the second polymer are the same as exemplified in relation to the solvent for dissolving the first polymer. The polymer concentration of the second polymer solution is, for example, 0.1 to 30 mass %, preferably 1 to 20 mass %.

No particular limitation is imposed on the method of bringing the second polymer solution into contact with the film-shape solid containing the first polymer. Examples of the method of bringing the second polymer solution into contact with the film-shape solid include applying (via dropwise addition or spraying) the second polymer solution onto the surface of the film-shape solid; and immersing the films-shape solid in a second polymer solution. In one preferred embodiment, a second polymer solution is applied (e.g., via dropwise addition) onto the surface of the film-shape solid, to thereby provide a liquid layer formed of the second polymer solution on the film-shape solid, and the product is allowed to stand for a predetermined time (e.g., 10 to 180 minutes). No particular limitation is imposed on the thickness of the liquid layer, and the thickness is, for example, 0.1 to 50,000 μm. Through the above procedure, the first polymer contained in the film-shape solid is gradually dissolved in the second polymer solution, to thereby form a hydrogel.

In the case where the second polymer solution is brought into contact with the film-shape solid containing the first polymer, the amount of the second polymer solution which is to be in contact with the film-shape solid is preferably adjusted such that a cross-link structure is suitably formed in the formed hydrogel. Specifically, the amounts and polymer concentrations of the film-shape solid and the second polymer solution are preferably controlled, such that the amount of the functional group E present in the polymer (B) with respect to 1 mol of carboxyl groups present in the polymer (A) is preferably 0.1 to 10 mol, more preferably 0.2 to 8 mol, still more preferably 0.5 to 2 mol.

When a dry product containing the water-soluble polymer (C) as the medical treatment material is provided, the water-soluble polymer (C) may be contained in the film-shape solid, or in the second polymer solution. In one possible case where the water-soluble polymer (C) is present in the second polymer solution, the water-soluble polymer (C) is incorporated in advance into the second polymer solution, and the resultant solution (i.e., the second polymer solution containing the water-soluble polymer (C)) is brought into contact with the film-shape solid. In an alternative procedure, the second polymer solution is brought into contact with the film-shape solid, and then the water-soluble polymer (C) is added to the second polymer solution. From the viewpoint of suitably forming a hydrogel, among these procedures, the mode of the second polymer solution containing the water-soluble polymer (C) is preferred. More preferably, the second polymer solution containing in advance the water-soluble polymer (C) is brought into contact with the film-shape solid.

When the second polymer solution containing the water-soluble polymer (C) is brought into contact with the film-shape solid, the water-soluble polymer (C) content of the second polymer solution is preferably adjusted to 0.01 to 50 parts by mass with respect to 100 parts by mass of the second polymer, more preferably to 0.1 to 20 parts by mass, still more preferably to 0.5 to 15 parts by mass.

Thereafter, the thus-obtained hydrogel is dried, to thereby yield a target dry product. No particular limitation is imposed on the method of drying the hydrogel, and any known drying technique may be appropriately employed.

When drying of hydrogel is performed through solution drying, freeze drying is preferably employed. In freeze drying, the cooling temperature for freezing is, for example, −70° C. to −5° C., preferably −60° C. to −5° C. Drying treatment through freeze drying is preferably performed at room temperature under reduced pressure. The pressure at which freeze drying is performed is, for example, 50 Pa or lower, preferably 20 Pa or lower, more preferably 10 Pa or lower. As used herein, the term “dry” refers to a state in which water has been completely removed and, alternatively, a state in which water remains after a drying step. The water content is a dried product obtained through drying is, for example, 10 mass % or less, preferably 5 mass % or less. In the case where the dried product is a film, the thickness of the film is, for example, 0.1 to 50,000 m. Through the above procedure, there can be produced a dry product which is in a state where a film-shape solid containing one of the polymer (A) and the polymer (B) has been brought into contact with a solution containing the other of them and the contact product is dried.

### (Method [2])

In the method [2], a solution containing the polymer (A) is mixed with a solution containing the polymer (B) in the presence of the water-soluble polymer (C), and the liquid mixture is dried, to thereby produce a medical treatment material as a dry product.

Regarding a solution containing the polymer (A) (hereinafter may also be referred to as a “polymer solution A”) and a solution containing the polymer (B) (hereinafter may also be referred to as a “polymer solution B”), examples of the solvent for dissolving a relevant polymer are the same as exemplified in relation to the solvent for dissolving the first polymer. Among them, water is preferably used as a sole component, from the viewpoint of efficiently conducting a drying step. The polymer concentration of each of the polymer solution A and the polymer solution B is, for example, 0.001 to 5 mass %, preferably 0.01 to 1 mass %.

Also, regarding the polymer (A) content and the polymer (B) content of the polymer solution A and the polymer solution B, the polymer solution A amount and concentration and the polymer solution B amount and concentration are preferably controlled such that the amount of the polymer (B) is adjusted to 20 to 500 parts by mass with respect to 100 parts by mass of the polymer (A). The amount of polymer (B) with respect to 100 parts by mass of the polymer (A) is more preferably adjusted to 30 to 400 parts by mass, still more preferably to 50 to 300 parts by mass.

Examples of the water-soluble polymer (C) employed in the method [2] are the same as specifically exemplified in relation to the water-soluble polymer (C) above. Among them, hyaluronic acid and a salt thereof are preferably used. The amount of the water-soluble polymer (C) with respect to 100 parts by mass of the polymer (A) is preferably adjusted to 0.01 to 50 parts by mass, more preferably to 0.1 to 20 parts by mass, still more preferably to 0.5 to 15 parts by mass. The water-soluble polymer (C) is preferably used in the form of aqueous solution.

Subsequently, a liquid mixture containing the polymer (A), the polymer (B), and the water-soluble polymer (C) prepared above is subjected to drying, to thereby yield a target dry product. Drying is preferably performed via freeze drying. Freeze drying may be performed through a customary method. In one typical procedure, the aforementioned liquid mixture is placed into a mold and frozen, and the molded frozen product is freeze-dried, to thereby yield a target product (dry product) of a shape on interest. The water content of the dry product is, for example, 10 mass % or less, preferably 5 mass % or less.

### <Embodiment of Use of Medical Treatment Material>

Before use, the medical treatment material of the present disclosure is a dry solid (i.e., a dry product). Upon contact with water, the material absorbs water and swells, to thereby provide a hydrogel (i.e., a swelled product). Before contact with water, the medical treatment material of the present disclosure is a dry product having flexibility. The dry product becomes a swelled product through contact with water, to thereby exhibit an adhesion property to a biotissue. Examples of water component include water, an organic solvent dissolvable in water (e.g., ethanol), a body fluid (e.g., blood or tissue fluid), and a liquid mixture thereof. The medical treatment material of the present disclosure is not absorbed by an organism (living body) and gradually decomposes under physiological conditions, whereby the material is solubilized. Thus, the material is highly safe and can be left in a living body. The medical treatment material having such characteristics is particularly suitable for various medical treatment materials such as an anti-adhesive material (for preventing synechia), a hemostatic material, and a wound dressing material.

No particular limitation is imposed on the shape of the medical treatment material of the present disclosure, and the material may be used as a film product, a sponge product, a sheet product, a powder product, etc. The medical treatment material of the present disclosure may be provided while held on a support or while included in a package (e.g., film). No particular limitation is imposed on the shape and material of the support. Examples of the support include fabric such as woven or nonwoven fabric; and resin substrates such as polystyrene, polypropylene, and polyethylene. The medical treatment material of the present disclosure, which has high dynamic strength and excellent flexibility, is particularly suitably used as, among others, a hydrogel-forming film or sponge.

### EXAMPLES

The disclosure will be described more specifically by way of example, which should not be construed as limiting the disclosure thereto. Unless otherwise specified, the units “part(s)” and “%” refer to “part(s) by mass” and “mass %,” respectively.

### <Production of Polymers>

### Synthesis Example 1: Synthesis of Polymer A

To a four-neck flask (capacity: 1 L), acrylic acid (purity: 99.9 mass % or more) (85 parts by mass), acrylic acid dimer (β-carboxyethyl acrylate, product of SIGMA-ALDRICH, product name: “2-carboxyethyl acrylate”) (0.26 parts by mass), pentaerythritol triallyl ether (0.4 parts by mass), n-hexane (200 parts by mass), and ethyl acetate (200 parts by mass) were added. The mixture was thoroughly degassed through bubbling with nitrogen gas, and the temperature of the mixture was elevated to 60° C. Then, polymerization was initiated by adding 2,2′-azobis(2,4-dimethylvaleronitrile) (0.03 parts by mass) to the mixture. Ten hours after the start of polymerization, cooling of the polymerization reaction mixture was started. When the temperature of the mixture was lowered to 25° C., a reaction mixture containing a polymer was recovered. The reaction mixture was dried under reduced pressure at 100° C. for 24 hours or longer, to thereby remove volatile components. Thus, a polymer of interest (hereinafter may also be referred to as a “polymer A”) was yielded.

### Synthesis Example 2: Synthesis of Polymer B

The procedure of Synthesis Example 1 was repeated, except that the amounts of monomers fed to the flask were changed; i.e., acrylic acid (purity: 99.9 mass % or more) (85 parts by mass), acrylic acid dimer (β-carboxyethyl acrylate, product of SIGMA-ALDRICH, product name: “2-carboxyethyl acrylate”) (0.86 parts by mass), and pentaerythritol triallyl ether (0.4 parts by mass), to thereby yield a polymer B.

### Synthesis Example 3: Synthesis of Polymer C

The procedure of Synthesis Example 1 was repeated, except that the amounts of monomers fed to the flask were changed; i.e., methacrylic acid (76.5 parts by mass), methyl methacrylate (8.5 parts by mass), and pentaerythritol triallyl ether (0.4 parts by mass), and that the amount of the polymerization initiator fed to the flask was changed; i.e., 2,2′-azobis(2,4-dimethylvaleronitrile) (0.06 parts by mass), to thereby yield a polymer C.

### Synthesis Example 4: Synthesis of Polymer D

The procedure of Synthesis Example 1 was repeated, except that the amounts of monomers fed to the flask were changed; i.e., methacrylic acid (68 parts by mass), methyl methacrylate (17 parts by mass), and pentaerythritol triallyl ether (0.4 parts by mass), and that the amount of the polymerization initiator fed to the flask was changed; i.e., 2,2′-azobis(2,4-dimethylvaleronitrile) (0.06 parts by mass), to thereby yield a polymer D.

### Synthesis Example 5: Synthesis of Polymer E

The procedure of Synthesis Example 1 was repeated, except that the amounts of monomers fed to the flask were changed; i.e., methacrylic acid (51 parts by mass), methyl methacrylate (34 parts by mass), and pentaerythritol triallyl ether (0.4 parts by mass), and that the amount of the polymerization initiator fed to the flask was changed; i.e., 2,2′-azobis(2,4-dimethylvaleronitrile) (0.06 parts by mass), to thereby yield a polymer E.

### Synthesis Example 6: Synthesis of Polymer F

The procedure of Synthesis Example 1 was repeated, except that the amounts of monomers fed to the flask were changed; i.e., acrylic acid (purity: 99.9 mass % or more) (76.5 parts by mass), ω-carboxy-caprolactone mono(meth)acrylate (n=about 2) (product of TOAGOSEI Co., Ltd., ARONIX (registered trademark) M-5300) (8.5 parts by mass, and pentaerythritol triallyl ether (0.4 parts by mass), to thereby yield a polymer F.

### Synthesis Example 7: Synthesis of Polymer G

To a four-neck flask (capacity: 1 L), acrylamide (85 parts by mass), n-butyl acrylate (15 parts by mass), pentaerythritol triallyl ether (0.4 parts by mass), n-hexane (200 parts by mass), and ethyl acetate (200 parts by mass) were added to obtain a mixture. The mixture was thoroughly degassed through bubbling with nitrogen gas, and the temperature of the mixture was elevated to 60° C. Then, polymerization was initiated by adding 2,2′-azobis(2,4-dimethylvaleronitrile) (0.03 parts by mass) to the mixture. Ten hours after the start of polymerization, cooling of the polymerization reaction mixture was started. When the temperature of the mixture was lowered to 25° C., a reaction mixture containing a polymer was recovered. The reaction mixture was dried under reduced pressure at 100° C. for 24 hours or longer, to thereby remove volatile components. Thus, a polymer of interest (hereinafter may also be referred to as a “polymer G”) was yielded.

### <Production of Hydrogel-Forming Sponge>

### Example 1

On a polypropylene support (50 mm×50 mm), a silicone rubber sheet (thickness: 10 mm) having an opening (25 mm×7 mm) was placed. A 1.2% aqueous solution of polymer A (1.5 mL) was cast thereonto and dried at 70° C. for 20 hours, to thereby produce a film of polymer A. Subsequently, a solution mixture of a 4.6% aqueous solution of polyvinylpyrrolidone (hereinafter may also be referred to as “PVP”) (0.6 mL) and a 0.4% aqueous solution of sodium hyaluronate (hereinafter may also be referred to as “HA”) (0.9 mL) was added dropwise to the surface of the polymer A film. The product was allowed to stand for 60 minutes and then frozen at −50° C. The frozen product was freeze-dried at room temperature under reduced pressure (5 Pa), to thereby provide a hydrogel-forming sponge (dimensions: 25 mm×7 mm×7 mm) as a medical treatment material. The proportions among the components: polymer A:PVP:HA were set to 1:1.53:0.2 (by mass).

### Examples 2 to 9 and Comparative Example 1

The procedure of Example 1 was repeated, except that the type of the raw materials were changed to the values specified in Table 1, to thereby provide hydrogel-forming sponges as medical treatment materials.

### <Method of Assessment>

Each of the hydrogel-forming sponges of Examples 1 to 9 and Comparative Example 1 was assessed in terms of adhesion force to a biotissue (skin). The procedure of evaluation was as follows. Table 1 shows the results.

### Measurement and Assessment of Adhesion Force to Biotissue (Skin)

Protein leather (Protein Leather PBZ13001-BK, product of IDEATEX JAPAN) was used as an artificial skin sample. The plane adhesion strength of the hydrogel-forming sponge to the protein leather sample was measured. In a specific procedure, a protein leather square piece (3 cm×3 cm) was adhered onto a cap of a centrifuge tube (50 mL) by use of an instantaneous adhesive (Aron Alpha (registered trademark), product of TOAGOSEI Co., Ltd.), and two pieces of this configuration were prepared. An appropriate amount of water was applied with a cotton swab onto the surface of each protein leather piece. Then, a hydrogel-forming sponge sample was sandwiched with two protein leather pieces. A weight of 300 g was placed on the prepared test sample, and the test sample was allowed to stand for 1 minute. Then, the weight was removed. One minute thereafter, tensile force was applied to the sample at 25° C. and 120 mm/mmn by means of a tensile tester. The maximum stress generated upon the tensile test was measured.

TABLE 1

| | Medical Treatment Material | | | Assessment |
|---|---|---|---|---|
| | | | Water- | Adhesion |
| | | | soluble | force to skin |
| | Polymer (A) | Polymer (B) | polymer (C) | (N/cm²) |
| Example 1 | Polymer A | PVP | HA | 3.0 |
| | (AA/Acrylic acid dimer = | (VP = 100 wt %) | | |
| | 99.7/0.3 wt %) | | | |
| Example 2 | Polymer B | PVP | HA | 4.1 |
| | (AA/Acrylic acid dimer = | (VP = 100 wt %) | | |
| | 99.0/1.0 wt %) | | | |
| Example 3 | Polymer C | PVP | HA | 4.2 |
| | (MAA/MMA = 90/10 wt %) | (VP = 100 wt %) | | |
| Example 4 | Polymer D | PVP | HA | 3.8 |
| | (MAA/MMA = 80/20 wt %) | (VP = 100 wt %) | | |
| Example 5 | Polymer E | PVP | HA | 3.0 |
| | (MAA/MMA = 60/40 wt %) | (VP = 100 wt %) | | |
| Example 6 | Polymer F | PVP | HA | 3.5 |
| | (AA/M-5300 = 90/10 wt %) | (VP = 100 wt %) | | |
| Example 7 | PAA | Polymer G | HA | 3.1 |
| | (AA = 100 wt %) | (AAm/BA = 85/15 wt %) | | |
| Example 8 | Polymer B | Polymer G | HA | 3.5 |
| | (AA/Acrylic acid dimer = | (AAm/BA = 85/15 wt %) | | |
| | 99.0/1.0 wt %) | | | |
| Example 9 | Polymer B | PVP | | 3.7 |
| | (AA/Acrylic acid dimer = | (VP = 100 wt %) | | |
| | 99.0/1.0 wt %) | | | |
| Comparative | PAA | PVP | HA | 2.5 |
| Example 1 | (AA = 100 wt %) | (VP = 100 wt %) | | |

The compounds employed in the test and shown in Table 1 are as follows.
AA: acrylic acid
MAA: methacrylic acid
MMVA: methyl methacrylate
M-5300: ω-carboxy-caprolactone mono(meth)acrylate (n=about 2) (product of TOAGOSEI Co., Ltd., ARONIX (registered trademark) M-5300)
VP: N-vinyl-2-pyrrolidone
AAm: acrylamide
BA: n-butyl acrylate
PAA: cross-linked poly(acrylic acid) (Carbopol 934P NF, product of Lubrizol)
PVP: polyvinylpyrrolidone (Kollidon 90F, product of BASF, weight average molecular weight (reduced to polystyrene=320,000 (eluent: dimethylformamide)
HA: sodium hyaluronate (Hiaruronsan HA-LQH, product of Kewpie Corporation)

### <Assessment Results>

As is clear from Table 1, the hydrogel-forming sponge samples of Examples 1 to 9, in which at least one of the polymer (A) and the polymer (B) included a structural unit derived from the ethylenic unsaturated monomer (mc), exhibited an adhesion force value to the skin as high as 3.0 N/cm² or more, indicating excellent handleability. Among these sponge samples, the samples each including a structural unit derived from a carboxyl group-containing ethylenic unsaturated monomer having a molecular weight of more than 115 (i.e., acrylic acid dimer or ω-carboxy-caprolactone mono(meth)acrylate) as the ethylenic unsaturated monomer (mc), the adhesion force to the skin was found to be enhanced, even when the ethylenic unsaturated monomer (mc) content was relatively small. In addition, from the results of comparative studies of Example 4 with Example 7, and the results of Example 8, the adhesion force to the skin was found to be enhanced in the case where a structural unit derived from the ethylenic unsaturated monomer (mc) was more incorporated into the polymer (A), as compared with the polymer (B).

In contrast, the sponge samples in which any of the polymer (A) and the polymer (B) include no structural unit derived from the ethylenic unsaturated monomer (mc) exhibited unsatisfactory adhesion force of the hydrogel to the skin, indicating insufficient adhesion performance (Comparative Example 1).

The present invention is not limited to the aforementioned embodiments. Needless to say, the present invention encompasses various modifications and those falling within the equivalents thereof, so long as they are not deviated from the gist of the present invention. Thus, it should be construed that, in view of the above teaching, those skilled in the art could conceive various combinations, modes, and further embodiments of a single element or a combination including the element or its equivalent, which also fall within the scope or concept of the present invention.

1. A medical treatment material that forms a hydrogel upon contact with water comprising:
a polymer (A) comprising a structural unit derived from an ethylenic unsaturated monomer (ma) which has a carboxyl group and a molecular weight of 115 or less, and
a polymer (B) comprising a structural unit derived from an ethylenic unsaturated monomer (mb) which has a functional group E that is capable of forming a hydrogen bond with a carboxyl group, the polymer (B) not being the polymer (A),
wherein at least one of the polymer (A) and the polymer (B) further comprises a structural unit derived from an ethylenic unsaturated monomer (mc) differing from the ethylenic unsaturated monomers (ma) and (mb), and the monomer (me) is not an ethylenic unsaturated monomer having a cross-linkable functional group.

2. The medical treatment material according to claim 1, wherein the ethylenic unsaturated monomer (mc) comprises an ethylenic unsaturated monomer (mc1) which has a carboxyl group and a molecular weight of more than 115.

3. The medical treatment material according to claim 2, wherein the ethylenic unsaturated monomer (mc1) is at least one species selected from the group consisting of an acrylic acid dimer, an acrylic acid trimer, an acrylic acid tetramer, and ω-carboxy-caprolactone mono(meth)acrylate.

4. The medical treatment material according to claim 1, wherein the ethylenic unsaturated monomer (mc) comprises an ethylenic unsaturated monomer (mc2) which has no carboxyl group and no functional group E.

5. The medical treatment material according to claim 1, wherein the polymer (A) comprises a structural unit derived from the ethylenic unsaturated monomer (mc), and has a ratio by mass ((ma)/(mc)) of the ethylenic unsaturated monomer (ma) to the ethylenic unsaturated monomer (mc), each forming the polymer (A), of 99.7/0.3 to 50/50.

6. The medical treatment material according to claim 1, wherein the polymer (A) is a cross-linked polymer.

7. The medical treatment material according to claim 1, wherein the polymer (B) has an amide group.

8. The medical treatment material according to claim 1, wherein the polymer (B) is at least one species selected from the group consisting of polyvinylpyrrolidone, polyacrylamide, and polymethacrylamide.

9. The medical treatment material according to claim 1,
which is in a state where a film-shape solid comprising one of the polymer (A) and the polymer (B) has been brought into contact with a solution comprising the other of the polymer (A) and the polymer (B) and a contact product is dried;
which forms a hydrogel upon absorption of water, and which exhibits an adhesion property to biotissue.

10. A method for producing a medical treatment material for forming a hydrogel upon contact with water, the production method comprising:
preparing a film-shape solid comprising one of a polymer (A) and a polymer (B) and
bringing a solution comprising the other of the polymer (A) and polymer (B) into contact with the film-shape solid, followed by drying,
wherein
the polymer (A) comprises a structural unit derived from an ethylenic unsaturated monomer (ma) which has a carboxyl group and a molecular weight of 115 or less;
the polymer (B) comprises a structural unit derived from an ethylenic unsaturated monomer (mb) which has a functional group E that is capable of forming a hydrogen bond with a carboxyl group, the polymer (B) not being the polymer (A); and
at least one of the polymer (A) and the polymer (B) further comprises a structural unit derived from an ethylenic unsaturated monomer (mc) differing from the ethylenic unsaturated monomers (ma) and (mb), and the monomer (me) is not an ethylenic unsaturated monomer having a cross-linkable functional group.

11. The method for producing the medical treatment material according to claim 10, wherein the ethylenic unsaturated monomer (mc) comprises an ethylenic unsaturated monomer (mc1) which has a carboxyl group and a molecular weight of more than 115.

12. The method for producing the medical treatment material according to claim 11, wherein the ethylenic unsaturated monomer (mc1) is at least one species selected from the group consisting of an acrylic acid dimer, an acrylic acid trimer, an acrylic acid tetramer, and ω-carboxy-caprolactone mono(meth)acrylate.

13. The method for producing the medical treatment material according to claim 10, wherein the ethylenic unsaturated monomer (mc) is an alkyl (meth)acrylate ester in which an alkyl group of an alkyl ester moiety has 1 to 4 carbon atoms.

14. The medical treatment material according to claim 4, wherein the ethylenic unsaturated monomer (mc2) is an alkyl (meth)acrylate ester in which an alkyl group of an alkyl ester moiety has 1 to 4 carbon atoms.

15. The medical treatment material according to claim 1, wherein the polymer (B) is a cross-linked polymer or a polymer having a weight average molecular weight of 10,000 or more.
